(19) United States
(12) Patent Application Publication
(10) Pub. No.: US 2026/0210813 A1
(43) Pub. Date: Jul. 23, 2026

(54) METHOD FOR EXTRACTING VOLATILE MATERIAL FROM MOON SOIL, AND GAS COLLECTION SYSTEM USING THE SAME

(71) Applicant: Korea Institute Of Geoscience And Mineral Resources, Daejeon (KR)

(72) Inventor: Kyeong Ja KIM, Daejeon (KR)

(73) Assignee: Korea Institute Of Geoscience And Mineral Resources, Daejeon (KR)

(21) Appl. No.: 19/282,559

(22) Filed: Jul. 28, 2025

(30) Foreign Application Priority Data
Aug. 9, 2024 (KR) 10-2024-0106783

(51) Int. Cl. G01N 1/22; F24S 20/30; F24S 23/70; H01J 49/42

(52) U.S. Cl. CPC G01N 1/2294; F24S 20/30; F24S 23/82; G01N 1/2205; H01J 49/4215

(57) ABSTRACT

A method for extracting volatile material from moon soil and a gas collection system using the same are provided, in which frozen soil is collected from the lunar regolith, heated by a solar heat collector, and volatile materials are separated and stored from the soil.

### CROSS-REFERENCE TO RELATED APPLICATION

This application claims priority to Korean Patent Application No. 10-2024-0106783 filed on Aug. 9, 2024, and all the benefits accruing therefrom under 35 U.S.C. § 119, the contents of which is incorporated by reference in its entirety.

### BACKGROUND OF THE DISCLOSURE

### 1. Field of the Disclosure

The present disclosure relates to a method for extracting volatile material from moon soil and a gas collection system using the same, in which frozen soil is collected from the lunar regolith, heated by a solar heat collector, and volatile materials are separated and stored from the soil.

### 2. Description of the Related Art

It is necessary for humankind to advance into space in order to survive, and the Moon and Mars are drawing attention as targets for space exploration.

The Moon is considered a forward base for reaching Mars, and efforts are being made to advance to Mars by acquiring space resources from the Moon.

The space resources to be acquired from the Moon include hydrogen, oxygen, water, helium, and carbon series gases.

Accordingly, through prolonged efforts and extensive research, the applicant of the present disclosure has completed the invention by acquiring a method for extracting volatile material from moon soil and a gas collection system using the same, in which frozen soil is collected from the lunar regolith, heated by a solar heat collector, and volatile materials are separated and stored from the soil.

### Related Patent Document

U.S. Pat. No. 11,719,100 (Aug. 8, 2023)

### SUMMARY OF THE DISCLOSURE

Therefore, the purpose of the present disclosure is to provide a method for extracting volatile material from moon soil, in which frozen soil is collected from the lunar regolith, heated by a solar heat collector, and volatile materials are separated and stored from the soil.

In addition, the purpose of the present disclosure is to provide a gas collection system using the method for extracting volatile material from moon soil.

The challenges that the present disclosure is intended to solve are not limited to those mentioned above, and other challenges not mentioned will be apparent to those skilled in the art from the following description.

In order to achieve the purpose, an aspect of the present disclosure provides a method for extracting volatile material from moon soil, comprising the steps of:
(a-1) collecting frozen soil from lunar regolith, heating the soil by a solar heat collector, extracting mixed gas from the soil, and storing the mixed gas;
(a-2) performing primary analysis and separation of the mixed gas, and storing hydrogen, oxygen, and water; and
(a-3) after performing the primary analysis and separation of the mixed gas, separating and storing noble gas series gases including at least one gas selected from the group consisting of helium-3 and helium-4, and carbon series gases,
wherein the soil is obtained from a permanently shadowed region of the Moon.

In some exemplary embodiments, in the step (a-1) of collecting frozen soil from lunar regolith, heating the soil by a solar heat collector, extracting mixed gas from the soil, and storing the mixed gas,
the solar heat collector may be configured as a primary heat exchanger that converts solar energy into thermal energy and transfers the thermal energy to a medium, the solar heat collector including a reflector, a transparent cover, and an insulator.

In some exemplary embodiments, the reflector of the solar heat collector may be a reflector made of aluminum, glass, or tempered glass for solar heat collection.

In some exemplary embodiments, in the step (a-1) of collecting frozen soil from lunar regolith, heating the soil by a solar heat collector, extracting mixed gas from the soil, and storing the mixed gas,
when the frozen soil is heated by the solar heat collector, the frozen soil may be heated such that gas contained in pores of the soil or in constituent materials of the soil is heated to be extracted as mixed gas, and the extracted mixed gas may be stored in a chamber or a container.

In some exemplary embodiments, in the step (a-1) of collecting frozen soil from lunar regolith, heating the soil by a solar heat collector, extracting mixed gas from the soil, and storing the mixed gas,
the mixed gas may comprise at least one gas selected from the group consisting of hydrogen series gases, helium series gases, carbon series gases, nitrogen series gases, oxygen series gases, sulfur series gases, and chlorine series gases.

In some exemplary embodiments, in the step (a-2) of performing primary analysis and separation of the mixed gas, and storing hydrogen, oxygen, and water,
the mixed gas may be analyzed by a mass spectrometer or a gas measurement device, and may be separated by a membrane filter, and
the mass spectrometer may be a quadrupole mass spectrometer.

In some exemplary embodiments, in the step (a-2) of performing primary analysis and separation of the mixed gas, and storing hydrogen, oxygen, and water,
the mixed gas may be primarily analyzed and separated to store hydrogen, oxygen, and water,
and the hydrogen may be further reacted with iron oxide in a reduction reaction to generate water additionally,
or alternatively, the hydrogen may be further reacted with FeTiO₃ in a reduction reaction to generate water additionally.

In some exemplary embodiments, in the step (a-3) of, after performing the primary analysis and separation of the mixed gas, separating and storing noble gas series gases including at least one gas selected from the group consisting of helium-3 and helium-4, and carbon series gases,
the mixed gas may be analyzed by a mass spectrometer or a gas measurement device, and is separated by a membrane filter,
wherein the mass spectrometer may be a quadrupole mass spectrometer.

In some exemplary embodiments, in the step (a-3) of, after performing the primary analysis and separation of the mixed gas, separating and storing noble gas series gases including at least one gas selected from the group consisting of helium-3 and helium-4, and carbon series gases,
the noble gas series gases may be liquefied after being separated, and stored in a chamber and a container.

In some exemplary embodiments, in the step (a-3) of, after performing the primary analysis and separation of the mixed gas, separating and storing noble gas series gases including at least one gas selected from the group consisting of helium-3 and helium-4, and carbon series gases,
the carbon series gases may comprise at least one selected from the group consisting of carbon monoxide, carbon dioxide, methanol, ethanol, methane, and ethane,
the carbon series gases may be stored in a chamber and a container after being separated,
and methane conversion may be further performed by adding hydrogen and oxygen to the carbon series gases.

In addition, another aspect of the present disclosure provides a gas collection system using a method for extracting volatile material from moon soil, comprising:
a raw material injection unit (110) configured to collect frozen soil from lunar regolith by a soil collector (103) and inject the soil into a soil heating chamber (107) through a soil injector (105);
a mixed gas storage unit (120) configured to heat the soil in the soil heating chamber (107) by a solar heat collector (111), extract mixed gas from the soil, and store the mixed gas;
a survival material storage unit (130, 133, 135) configured to perform primary analysis and separation of the mixed gas, and store hydrogen, water, and oxygen; and
a volatile material storage unit (150, 160) configured to, after performing primary analysis and separation of the mixed gas, separate and store noble gas series gases including at least one gas selected from the group consisting of helium-3 and helium-4, and carbon series gases.

In some exemplary embodiments, the soil may be obtained from a permanently shadowed region of the Moon,
wherein the soil collector (103) may be a module or a robotic system, and
wherein the soil injector (105) may be a module or a robotic system.

In some exemplary embodiments, the survival material storage unit (130, 133, 135) may be configured to store the hydrogen, the water, and the oxygen, respectively.

In some exemplary embodiments, the volatile material storage unit (150, 160) may be configured to store the noble gas series gases including at least one gas selected from the group consisting of helium-3 and helium-4, and the carbon series gases, respectively.

In addition, still another aspect of the present disclosure provides helium-3 and helium-4 recovered by the method for extracting volatile material from moon soil.

In addition, still another aspect of the present disclosure provides hydrogen, oxygen, and water recovered by the method for extracting volatile material from moon soil.

In addition, still another aspect of the present disclosure provides carbon series gases recovered by the method for extracting volatile material from moon soil.

In addition, still another aspect of the present disclosure provides helium-3 and helium-4 recovered by the gas collection system using the method for extracting volatile material from moon soil.

In addition, still another aspect of the present disclosure provides hydrogen, oxygen, and water recovered by the gas collection system using the method for extracting volatile material from moon soil.

In addition, still another aspect of the present disclosure provides carbon series gases recovered by the gas collection system using the method for extracting volatile material from moon soil.

According to the present disclosure, a method for extracting volatile material from moon soil is provided, in which frozen soil is collected from the lunar regolith, heated by a solar heat collector, and volatile materials are separated and stored from the soil, thereby enabling an innovative approach that makes lunar habitation possible by means of the volatile material extraction method.

Furthermore, the present disclosure provides a gas collection system using the method for extracting volatile material from moon soil, which is innovative, excellent, and applicable for the purpose of lunar habitation.

The effects of the present disclosure are not limited to the aforementioned effects and should be understood to include all effects that can be inferred from the configurations of the present disclosure described in the detailed description or the claims.

### BRIEF DESCRIPTION OF THE DRAWINGS

FIG. 1 is a process flow diagram of a method for extracting volatile material from moon soil according to an exemplary embodiment of the present disclosure.

FIG. 2 is a schematic view of a gas collection system using the method for extracting volatile material from moon soil according to an exemplary embodiment of the present disclosure.

### DETAILED DESCRIPTION OF THE DISCLOSURE

Hereinafter, exemplary embodiments of the present disclosure will be described in detail with reference to related drawings.

The advantages and features of the present disclosure, and methods of accomplishing those advantages and features, will become apparent upon reference to the exemplary embodiments described in detail with reference to the accompanying drawings.

However, the present disclosure is not limited by the exemplary embodiments disclosed herein, but will be embodied in many and various forms. Therefore, those exemplary embodiments are provided merely to make the present disclosure complete and to give a complete picture of the scope of the present disclosure to one of ordinary skill in the art to which the present disclosure belongs, and the present disclosure shall be defined by the scope of the claims.

Further, hereinafter, in describing the present disclosure, a detailed description of a configuration determined that may unnecessarily obscure the subject matter of the present disclosure, for example, a detailed description of a known technology including the prior art may be omitted.

Hereinafter, exemplary embodiments of the present disclosure will be described in detail.

### Method for Extracting Volatile Material From Moon Soil

According to an exemplary embodiment of the present disclosure, a method for extracting volatile material from moon soil is provided, in which frozen soil is collected from the lunar regolith, heated by a solar heat collector, and volatile materials are separated and stored from the soil.

The method for extracting volatile material from moon soil according to the present disclosure comprises:
(a-1) collecting frozen soil from lunar regolith, heating the soil by a solar heat collector, extracting mixed gas from the soil, and storing the mixed gas;
(a-2) performing primary analysis and separation of the mixed gas, and storing hydrogen, oxygen, and water; and
(a-3) after performing the primary analysis and separation of the mixed gas, separating and storing noble gas series gases including at least one gas selected from the group consisting of helium-3 and helium-4, and carbon series gases,
wherein the soil is obtained from a permanently shadowed region of the Moon.

The present disclosure provides a method for extracting volatile material from moon soil, in which frozen soil is collected from the lunar regolith, heated by a solar heat collector, and volatile materials are separated and stored from the soil, thereby offering an innovative solution that enables lunar habitation through the volatile material extraction method.

As humankind requires space exploration for its survival, the Moon and Mars are drawing attention as primary targets for such endeavors.

The Moon is considered a forward base for reaching Mars, with efforts being made to advance to Mars by acquiring space resources from the Moon.

The space resources to be acquired from the Moon include hydrogen, oxygen, water, helium, and carbon series gases.

Accordingly, through prolonged efforts and extensive research, the applicant has completed the present disclosure by developing a method for extracting volatile material from moon soil, in which frozen soil is collected from the lunar regolith, heated by a solar heat collector, and volatile materials are separated and stored from the soil, and by developing a gas collection system using the same.

Here, the method for extracting volatile material from moon soil according to the present disclosure may be a method for extracting volatile material from moon soil, in which frozen soil is collected from the lunar regolith, heated by a solar heat collector, and volatile materials are separated and stored from the soil.

Here, in the step (a-1) of collecting frozen soil from lunar regolith, heating the soil by a solar heat collector, extracting mixed gas from the soil, and storing the mixed gas,
the solar heat collector may be configured as a primary heat exchanger that converts solar energy into thermal energy and transfers the thermal energy to a medium, the solar heat collector including a reflector, a transparent cover, and an insulator.

In addition, the reflector of the solar heat collector may be a reflector made of aluminum, glass, or tempered glass for solar heat collection.

Further, in the step (a-1) of collecting frozen soil from lunar regolith, heating the soil by a solar heat collector, extracting mixed gas from the soil, and storing the mixed gas,
when the frozen soil is heated by the solar heat collector, the frozen soil may be heated such that gas contained in pores of the soil or in constituent materials of the soil is heated to be extracted as mixed gas, and the extracted mixed gas may be stored in a chamber or a container.

Here, the soil may be obtained from a permanently shadowed region of the Moon.

In addition, the soil may be obtained from the lunar regolith.

Moreover, in the step (a-1) of collecting frozen soil from lunar regolith, heating the soil by a solar heat collector, extracting mixed gas from the soil, and storing the mixed gas,
the mixed gas may comprise at least one gas selected from the group consisting of hydrogen series gases, helium series gases, carbon series gases, nitrogen series gases, oxygen series gases, sulfur series gases, and chlorine series gases.

For example, the mixed gas may include hydrogen, helium-3, helium-4, water, hydrogen sulfide, ammonia, sulfur dioxide, carbon monoxide, carbon dioxide, methanol, methane, ethanol, ethane, hydroxide ions, hydrogen chloride, and the like.

In addition, in the step (a-2) of performing primary analysis and separation of the mixed gas, and storing hydrogen, oxygen, and water,
the mixed gas may be analyzed by a mass spectrometer or a gas measurement device, and may be separated by a membrane filter, and
the mass spectrometer may be a quadrupole mass spectrometer.

Here, the mass spectrometer may be an analyzer configured to convert gas extracted by heating the lunar soil into gas-phase ions, and analyze the gas by measuring different mass-to-charge ratios of the formed gas-phase ions, thereby identifying the molecules contained in the gas.

Furthermore, the quadrupole mass spectrometer includes four parallel metal rods. A voltage of +(U−Vcos(wt)) is applied to one pair of opposing rods, and a voltage of −(U−Vcos(wt)) is applied to the other pair of rods, where U is a direct current (DC) voltage and V is an alternating current (AC) voltage. The voltage applied to the four parallel rods affects the trajectory of ions. Under given DC and AC voltages, only ions having a specific mass-to-charge (m/z) ratio pass through the quadrupole filter, while the remaining ions deviate from their path. By measuring the ions passing through the quadrupole filter according to various voltage conditions, a mass spectrum is obtained.

There are two methods of adjusting the voltage: one method involves varying the w value while keeping the U and V values fixed, and the other method involves varying the U and V values while keeping the w value fixed.

Moreover, the mixed gas may be separated by a membrane filter.

In addition, in the step (a-2) of performing primary analysis and separation of the mixed gas, and storing hydrogen, oxygen, and water,
the mixed gas may be primarily analyzed and separated to store hydrogen, oxygen, and water,
and the hydrogen may be further reacted with iron oxide in a reduction reaction to generate water additionally,
or alternatively, the hydrogen may be further reacted with FeTiO₃ in a reduction reaction to generate water additionally.

Here, when the hydrogen is reacted with iron oxide (FeO) in a reduction reaction to generate water additionally, the reaction can be represented by the following Reaction Formula (1):

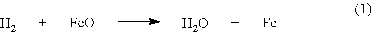

Furthermore, when the hydrogen is reacted with ilmenite (FeTiO₃) in a reduction reaction to generate water additionally, the reaction can be represented by the following Reaction Formula (2):

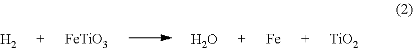

Furthermore, the water and hydrogen are separated, the hydrogen is reused for the next reduction reaction, and the water can be utilized for separating oxygen through electrolysis, with the hydrogen generated thereby being recycled again.

Here, as a method for producing water on the Moon, a hydrogen reduction reaction between the hydrogen and iron oxide or ilmenite (FeTiO₃) may be used.

In particular, water must be produced by heating the ilmenite (FeTiO₃), and hydrogen may be essential for the reaction.

Here, the hydrogen reduction reaction between the hydrogen and iron oxide or ilmenite (FeTiO₃) starts at approximately 900° C., and as the temperature increases up to approximately 1400° C., oxygen gradually increases due to water electrolysis, accounting for 25% or more of the total gas volume, while hydrogen accounts for approximately 75%.

In addition, the amount of oxygen extracted is proportional to the iron (II) ion content, and when the iron content is approximately 20%, approximately 5% oxygen production may be possible.

In addition, in the step (a-3) of, after performing the primary analysis and separation of the mixed gas, separating and storing noble gas series gases including at least one gas selected from the group consisting of helium-3 and helium-4, and carbon series gases,
the mixed gas may be analyzed by a mass spectrometer or a gas measurement device, and is separated by a membrane filter,
wherein the mass spectrometer may be a quadrupole mass spectrometer.

Further, in the step (a-3) of, after performing the primary analysis and separation of the mixed gas, separating and storing noble gas series gases including at least one gas selected from the group consisting of helium-3 and helium-4, and carbon series gases,
the noble gas series gases may be liquefied after being separated, and stored in a chamber and a container.

Moreover, in the step (a-3) of, after performing the primary analysis and separation of the mixed gas, separating and storing noble gas series gases including at least one gas selected from the group consisting of helium-3 and helium-4, and carbon series gases,
the carbon series gases may comprise at least one selected from the group consisting of carbon monoxide, carbon dioxide, methanol, ethanol, methane, and ethane,
the carbon series gases may be stored in a chamber and a container after being separated,
and methane conversion may be further performed by adding hydrogen and oxygen to the carbon series gases.

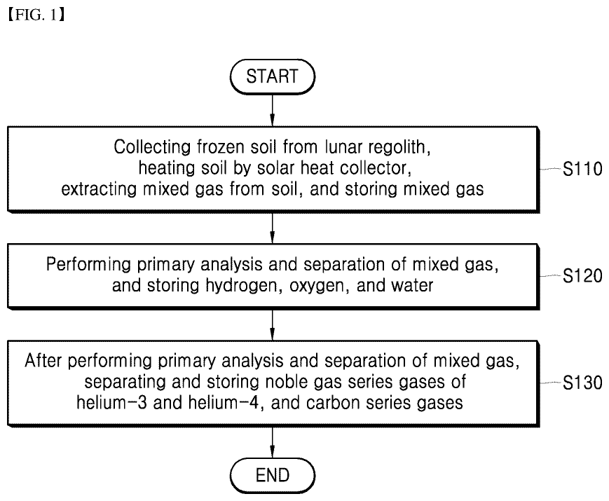

FIG. 1 is a process flow diagram of a method for extracting volatile material from moon soil according to an exemplary embodiment of the present disclosure.

Referring to FIG. 1, frozen soil is first collected from the lunar regolith, and the soil is heated by a solar heat collector to extract and store mixed gas from the soil (S110).

Thereafter, the mixed gas is subjected to primary analysis and separation, and hydrogen, oxygen, and water are stored (S120).

Then, after performing the primary analysis and separation of the mixed gas, noble gas series gases including at least one gas selected from the group consisting of helium-3 and helium-4, and carbon series gases are separated and stored (S130).

### Gas Collection System Using the Method for Extracting Volatile Material From Moon Soil

The present disclosure provides a gas collection system using a method for extracting volatile material from moon soil, in which frozen soil is collected from the lunar regolith, heated by a solar heat collector, and volatile materials are separated and stored from the soil.

The gas collection system (100) using the method for extracting volatile material from moon soil according to the present disclosure comprises:
a raw material injection unit (110) configured to collect frozen soil from lunar regolith by a soil collector (103) and inject the soil into a soil heating chamber (107) through a soil injector (105);
a mixed gas storage unit (120) configured to heat the soil in the soil heating chamber (107) by a solar heat collector (111), extract mixed gas from the soil, and store the mixed gas;
a survival material storage unit (130, 133, 135) configured to perform primary analysis and separation of the mixed gas, and store hydrogen, water, and oxygen; and
a volatile material storage unit (150, 160) configured to, after performing primary analysis and separation of the mixed gas, separate and store noble gas series gases including at least one gas selected from the group consisting of helium-3 and helium-4, and carbon series gases.

The present disclosure provides a gas collection system using a method for extracting volatile material from moon soil, in which frozen soil is collected from the lunar regolith, heated by a solar heat collector, and volatile materials are separated and stored from the soil, thereby offering an innovative and excellent solution that can be used for lunar habitation purposes.

In addition, the present disclosure may be a gas collection system using a method for extracting volatile material from moon soil, in which frozen soil is collected from the lunar regolith, heated by a solar heat collector, and volatile materials are separated and stored from the soil.

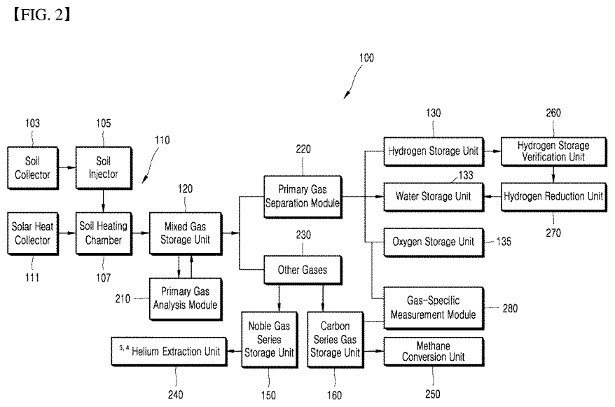

FIG. 2 is a schematic view of a gas collection system using the method for extracting volatile material from moon soil according to an exemplary embodiment of the present disclosure.

Referring to FIG. 2, the gas collection system (100) using a method for extracting volatile material from moon soil according to the present disclosure comprises:
a raw material injection unit (110) configured to collect frozen soil from lunar regolith by a soil collector (103) and inject the soil into a soil heating chamber (107) through a soil injector (105);
a mixed gas storage unit (120) configured to heat the soil in the soil heating chamber (107) by a solar heat collector (111), extract mixed gas from the soil, and store the mixed gas;
a survival material storage unit (130, 133, 135) configured to perform primary analysis and separation of the mixed gas, and store hydrogen, water, and oxygen; and
a volatile material storage unit (150, 160) configured to, after performing primary analysis and separation of the mixed gas, separate and store noble gas series gases including at least one gas selected from the group consisting of helium-3 and helium-4, and carbon series gases.

The gas collection system (100) using the method for extracting volatile material from moon soil may further include a primary gas analysis module (210) configured to perform primary analysis of the mixed gas stored in the mixed gas storage unit (120).

In addition, the gas collection system (100) using the method for extracting volatile material from moon soil may further include a primary gas separation module (220) configured to perform primary separation of the mixed gas stored in the mixed gas storage unit (120).

The gas collection system (100) using the method for extracting volatile material from moon soil may further separate the remaining other gases (230), after separating hydrogen, water, and oxygen in the primary gas separation module (220), into noble gas series gases and carbon series gases, and store them in the noble gas series gas storage unit (150) and the carbon series gas storage unit (160), respectively.

Furthermore, the gas collection system (100) using the method for extracting volatile material from moon soil may further include a helium extraction unit (240) configured to extract helium-3 and helium-4 from the noble gas series gas storage unit (150).

The gas collection system (100) using the method for extracting volatile material from moon soil may further include a gas-specific measurement module (280) configured to individually measure the carbon series gases stored in the carbon series gas storage unit (160).

In addition, the gas collection system (100) using the method for extracting volatile material from moon soil may further include a methane conversion unit (250) configured to convert the carbon series gases stored in the carbon series gas storage unit (160) into methane.

The gas collection system (100) using the method for extracting volatile material from moon soil may further include a hydrogen storage verification unit (260) configured to verify hydrogen storage in the hydrogen storage unit (130).

Here, the hydrogen storage verification unit (260) may transfer the hydrogen to a hydrogen reduction unit (270) to react the hydrogen with iron oxide or ilmenite, generate water additionally, and store the water in the water storage unit (133).

In addition, the soil may be obtained from a permanently shadowed region of the Moon,
wherein the soil collector (103) may be a module or a robotic system, and
wherein the soil injector (105) may be a module or a robotic system.

Here, the module of the soil collector (103) may be in the form of a scoop, and the robotic system may be a platform equipped with a heating device.

In addition, the module of the soil injector (105) may be connected by a conveyor belt in the case of a large-scale system. Alternatively, the module of the soil injector (105) may be in the form of a scoop in the case of a small-scale system. The robotic system may be a platform including the solar heat collector and the soil heating device.

In addition, the survival material storage unit (130, 133, 135) may be configured to store the hydrogen, the water, and the oxygen, respectively.

In this case, the survival material storage unit may include a hydrogen storage unit (130), a water storage unit (133), and an oxygen storage unit (135).

In addition, the volatile material storage unit (150, 160) may be configured to store the noble gas series gases including at least one gas selected from the group consisting of helium-3 and helium-4, and the carbon series gases, respectively.

Here, the volatile material storage unit may include a noble gas series storage unit (150) and a carbon series gas storage unit (160).

### Helium-3 and Helium-4 Recovered by the Method for Extracting Volatile Material From Moon Soil

The present disclosure provides helium-3 and helium-4 recovered by a method for extracting volatile material from moon soil, in which frozen soil is collected from the lunar regolith, heated by a solar heat collector, and volatile materials are separated and stored from the soil.

The present disclosure may provide helium-3 and helium-4 recovered by the method for extracting volatile material from moon soil.

Here, the helium-3 may serve as a clean nuclear fusion energy source and may also be used for research purposes, and the helium-4 may be used as a coolant.

Since the present disclosure provides helium-3 and helium-4 recovered by a method for extracting volatile material from moon soil, in which frozen soil is collected from the lunar regolith, heated by a solar heat collector, and volatile materials are separated and stored from the soil, the recovered helium-3 and helium-4 have excellent physical properties and may be used for various purposes such as enabling lunar habitation by humans.

### Hydrogen, Oxygen, and Water Recovered by the Method for Extracting Volatile Material From Moon Soil

The present disclosure provides hydrogen, oxygen, and water recovered by a method for extracting volatile material from moon soil, in which frozen soil is collected from the lunar regolith, heated by a solar heat collector, and volatile materials are separated and stored from the soil.

The present disclosure may provide hydrogen, oxygen, and water recovered by the method for extracting volatile material from moon soil.

Since the present disclosure provides hydrogen, oxygen, and water recovered by a method for extracting volatile material from moon soil, in which frozen soil is collected from the lunar regolith, heated by a solar heat collector, and volatile materials are separated and stored from the soil, the recovered hydrogen, oxygen, and water have excellent physical properties and may be used for various purposes, including enabling lunar habitation by humans.

### Carbon Series Gases Recovered by the Method for Extracting Volatile Material From Moon Soil

The present disclosure provides carbon series gases recovered by a method for extracting volatile material from moon soil, in which frozen soil is collected from the lunar regolith, heated by a solar heat collector, and volatile materials are separated and stored from the soil.

The present disclosure may provide carbon series gases recovered by the method for extracting volatile material from moon soil.

Here, the carbon series gases may be utilized according to each type of gas, wherein carbon dioxide may be used for plant cultivation, and other gases such as carbon monoxide may be used for producing methane gas as fuel.

Since the present disclosure provides carbon series gases recovered by a method for extracting volatile material from moon soil, in which frozen soil is collected from the lunar regolith, heated by a solar heat collector, and volatile materials are separated and stored from the soil, the recovered carbon series gases have excellent physical properties and may be used for various purposes, including enabling lunar habitation by humans.

### Helium-3 and Helium-4 Recovered by the Gas Collection System Using the Method for Extracting Volatile Material From Moon Soil

The present disclosure provides helium-3 and helium-4 recovered by a gas collection system using a method for extracting volatile material from moon soil, in which frozen soil is collected from the lunar regolith, heated by a solar heat collector, and volatile materials are separated and stored from the soil.

The present disclosure may provide helium-3 and helium-4 recovered by the gas collection system using the method for extracting volatile material from moon soil.

Here, the helium-3 may serve as a clean nuclear fusion energy source and may also be used for research purposes, and the helium-4 may be used as a coolant.

Since the present disclosure provides helium-3 and helium-4 recovered by a gas collection system using a method for extracting volatile material from moon soil, in which frozen soil is collected from the lunar regolith, heated by a solar heat collector, and volatile materials are separated and stored from the soil, the recovered helium-3 and helium-4 have excellent physical properties and may be used for various purposes, including enabling lunar habitation by humans.

### Hydrogen, Oxygen, and Water Recovered by the Gas Collection System Using the Method for Extracting Volatile Material From Moon Soil

The present disclosure provides hydrogen, oxygen, and water recovered by a gas collection system using a method for extracting volatile material from moon soil, in which frozen soil is collected from the lunar regolith, heated by a solar heat collector, and volatile materials are separated and stored from the soil.

The present disclosure may provide hydrogen, oxygen, and water recovered by the gas collection system using the method for extracting volatile material from moon soil.

Since the present disclosure provides hydrogen, oxygen, and water recovered by a gas collection system using a method for extracting volatile material from moon soil, in which frozen soil is collected from the lunar regolith, heated by a solar heat collector, and volatile materials are separated and stored from the soil, the recovered hydrogen, oxygen, and water have excellent physical properties and may be used for various purposes, including enabling lunar habitation by humans.

### Carbon Series Gases Recovered by the Gas Collection System Using the Method for Extracting Volatile Material From Moon Soil

The present disclosure provides carbon series gases recovered by a gas collection system using a method for extracting volatile material from moon soil, in which frozen soil is collected from the lunar regolith, heated by a solar heat collector, and volatile materials are separated and stored from the soil.

The present disclosure may provide carbon series gases recovered by the gas collection system using the method for extracting volatile material from moon soil.

Here, the carbon series gases may be utilized according to each type of gas; for example, carbon dioxide may be used for plant cultivation, and other gases such as carbon monoxide may be used for producing methane gas as fuel.

Since the present disclosure provides carbon series gases recovered by a gas collection system using a method for extracting volatile material from moon soil, in which frozen soil is collected from the lunar regolith, heated by a solar heat collector, and volatile materials are separated and stored from the soil, the recovered carbon series gases have excellent physical properties and may be used for various purposes, including enabling lunar habitation by humans.

In the above, exemplary embodiments of a method for extracting volatile material from moon soil and a gas collection system using the same according to the present disclosure have been described. Moreover, it will be appreciated that various modifications to these exemplary embodiments are possible without departing from the scope of the present disclosure.

The scope of the present disclosure should therefore not be limited to those exemplary embodiments described above, but should be defined by the following claims and their equivalents.

In other words, the foregoing exemplary embodiments are to be understood as illustrative rather than restrictive in all respects, and the scope of the present disclosure is indicated by the following claims rather than the detailed description. All modifications or variations derived from the meaning, scope, and equivalent concepts of the claims should be interpreted as being included within the scope of the present disclosure.

### List of Reference Numbers

100: gas collection system using the method for extracting volatile material from moon soil
103: soil collector
105: soil injector
107: soil heating chamber
110: raw material injection unit
111: solar heat collector
120: mixed gas storage unit
130: hydrogen storage unit
133: water storage unit
135: oxygen storage unit
150: noble gas series storage unit
160: carbon series gas storage unit
210: primary gas analysis module
220: primary gas separation module
230: other gases
240: helium-3 and helium-4 extraction unit
250: methane conversion unit
260: hydrogen storage verification unit
270: hydrogen reduction unit
280: gas-specific measurement module

What is claimed is:

1. A method for extracting volatile material from moon soil, comprising the steps of:
(a-1) collecting frozen soil from lunar regolith, heating the soil by a solar heat collector, extracting mixed gas from the soil, and storing the mixed gas;
(a-2) performing primary analysis and separation of the mixed gas, and storing hydrogen, oxygen, and water; and
(a-3) after performing the primary analysis and separation of the mixed gas, separating and storing noble gas series gases including at least one gas selected from the group consisting of helium-3 and helium-4, and carbon series gases,
wherein the soil is obtained from a permanently shadowed region of the Moon.

2. The method of claim 1,
wherein, in the step (a-1) of collecting frozen soil from lunar regolith, heating the soil by a solar heat collector, extracting mixed gas from the soil, and storing the mixed gas,
the solar heat collector is configured as a primary heat exchanger that converts solar energy into thermal energy and transfers the thermal energy to a medium, the solar heat collector including a reflector, a transparent cover, and an insulator.

3. The method of claim 2,
wherein the reflector of the solar heat collector is a reflector made of aluminum, glass, or tempered glass for solar heat collection.

4. The method of claim 1,
wherein, in the step (a-1) of collecting frozen soil from lunar regolith, heating the soil by a solar heat collector, extracting mixed gas from the soil, and storing the mixed gas,
when the frozen soil is heated by the solar heat collector, the frozen soil is heated such that gas contained in pores of the soil or in constituent materials of the soil is heated to be extracted as mixed gas, and the extracted mixed gas is stored in a chamber or a container.

5. The method of claim 1,
wherein, in the step (a-1) of collecting frozen soil from lunar regolith, heating the soil by a solar heat collector, extracting mixed gas from the soil, and storing the mixed gas,
the mixed gas comprises at least one gas selected from the group consisting of hydrogen series gases, helium series gases, carbon series gases, nitrogen series gases, oxygen series gases, sulfur series gases, and chlorine series gases.

6. The method of claim 1,
wherein, in the step (a-2) of performing primary analysis and separation of the mixed gas, and storing hydrogen, oxygen, and water,
the mixed gas is analyzed by a mass spectrometer or a gas measurement device, and is separated by a membrane filter,
wherein the mass spectrometer is a quadrupole mass spectrometer.

7. The method of claim 1,
wherein, in the step (a-2) of performing primary analysis and separation of the mixed gas, and storing hydrogen, oxygen, and water,
the mixed gas is primarily analyzed and separated to store hydrogen, oxygen, and water,
and the hydrogen is further reacted with iron oxide in a reduction reaction to generate water additionally,
or alternatively, the hydrogen is further reacted with FeTiO₃ in a reduction reaction to generate water additionally.

8. The method of claim 1,
wherein, in the step (a-3) of, after performing the primary analysis and separation of the mixed gas, separating and storing noble gas series gases including at least one gas selected from the group consisting of helium-3 and helium-4, and carbon series gases,
the mixed gas is analyzed by a mass spectrometer or a gas measurement device, and is separated by a membrane filter,
wherein the mass spectrometer is a quadrupole mass spectrometer.

9. The method of claim 1,
wherein, in the step (a-3) of, after performing the primary analysis and separation of the mixed gas, separating and storing noble gas series gases including at least one gas selected from the group consisting of helium-3 and helium-4, and carbon series gases,
the noble gas series gases are liquefied after being separated, and stored in a chamber and a container.

10. The method of claim 1,
wherein, in the step (a-3) of, after performing the primary analysis and separation of the mixed gas, separating and storing noble gas series gases including at least one gas selected from the group consisting of helium-3 and helium-4, and carbon series gases,
the carbon series gases comprise at least one selected from the group consisting of carbon monoxide, carbon dioxide, methanol, ethanol, methane, and ethane,
the carbon series gases are stored in a chamber and a container after being separated,
and methane conversion is further performed by adding hydrogen and oxygen to the carbon series gases.

11. A gas collection system using a method for extracting volatile material from moon soil, comprising:
a raw material injection unit (110) configured to collect frozen soil from lunar regolith by a soil collector (103) and inject the soil into a soil heating chamber (107) through a soil injector (105);
a mixed gas storage unit (120) configured to heat the soil in the soil heating chamber (107) by a solar heat collector (111), extract mixed gas from the soil, and store the mixed gas;
a survival material storage unit (130, 133, 135) configured to perform primary analysis and separation of the mixed gas, and store hydrogen, water, and oxygen; and
a volatile material storage unit (150, 160) configured to, after performing primary analysis and separation of the mixed gas, separate and store noble gas series gases including at least one gas selected from the group consisting of helium-3 and helium-4, and carbon series gases.

12. The gas collection system of claim 11,
wherein the soil is obtained from a permanently shadowed region of the Moon,
wherein the soil collector (103) is a module or a robotic system, and
wherein the soil injector (105) is a module or a robotic system.

13. The gas collection system of claim 11,
wherein the survival material storage unit (130, 133, 135) is configured to store the hydrogen, the water, and the oxygen, respectively.

14. The gas collection system of claim 11,
wherein the volatile material storage unit (150, 160) is configured to store the noble gas series gases including at least one gas selected from the group consisting of helium-3 and helium-4, and the carbon series gases, respectively.

15. Helium-3 and helium-4 recovered by the method of claim 1.

16. Hydrogen, oxygen, and water recovered by the method of claim 1.

17. Carbon series gases recovered by the method of claim 1.

18. Helium-3 and helium-4 recovered by the gas collection system of claim 11.

19. Hydrogen, oxygen, and water recovered by the gas collection system of claim 11.

20. Carbon series gases recovered by the gas collection system of claim 11.
